(19) United States
(12) Patent Application Publication
(10) Pub. No.: US 2021/0402208 A1
(43) Pub. Date: Dec. 30, 2021

(54) DEVICE AND METHOD FOR CONTROLLING TREATMENT FOR A SKIN CONDITION USING A TRACER

(71) Applicant: JOHNSON & JOHNSON CONSUMER INC., SKILLMAN, NJ (US)

(72) Inventor: Albert Durr EDGAR, Austin, TX (US)

(21) Appl. No.: 17/305,019

(22) Filed: Jun. 29, 2021

Related U.S. Application Data
(60) Provisional application No. 63/046,521, filed on Jun. 30, 2020.

(51) Int. Cl. A61N 5/06; G06T 7/00; A61K 8/18; A61K 49/00

(52) U.S. Cl. CPC A61N 5/0616; G06T 7/0012; A61K 8/18; A61N 2005/0644; G06T 2207/30088; G06T 2207/20182; A61N 2005/066; A61K 49/0032

(57) ABSTRACT

A device and method for treating a skin condition is provided. The device may include an applicator arrangement applying a composition to skin. The composition comprising an active ingredient for treating the skin condition and a tracer. The device may also include a detector arrangement obtaining image data corresponding to an image of an area of skin. The device may further include a processing arrangement analyzing the image data to determine whether an artifact corresponding to the skin condition is detected at a location within the imaged area of the skin and to determine an amount of the tracer detected from the location, the processing arrangement directing the applicator arrangement to apply the composition to the location when the artifact is detected and the amount of the tracer is less than a predetermined threshold level.

### PRIORITY CLAIM

This application claims priority to U.S. Provisional Application Ser. No. 63/046,521 filed Jun. 30, 2020, the entire contents of which is hereby incorporated by reference herein.

### FIELD OF INVENTION

The present invention relates to devices and methods for treating a condition on a treatment surface, such as a keratinous surface (e.g., the skin, hair or nails) or enamel (e.g., teeth). More specifically, the invention relates to devices and methods for selectively applying a treatment to the surface and tracking said treatment using a tracer.

### BACKGROUND

Manual application of topical skin treatments relies on visual inspection of the skin and manual administration of the topical skin treatments by the user. This way of administrating a topical skin treatment relies on visual identification of those areas of skin in need of treatment. However, a skin condition may be present before any visible symptoms are observed on the skin by the eye. Therefore, such visual inspection and manual application of topical skin treatments are ineffective at identifying areas of the skin suffering from a skin condition but has yet to form visible skin artifacts. In addition, manual administration of the topical treatment may result in application of the skin treatment to a larger area of skin than those concise areas that suffer from the skin condition. For example, a user may manually apply a topical ointment using a finger, which is typically wider than the concise areas of skin artifacts, such as, for example, acne outbreaks, pre-emergent acne or hyperpigmentation in need of treatment. Such broad application of a treatment is undesirable because the treatment can be associated with potential adverse effects. Furthermore, the treatment is not expected to impart any beneficial effects to those areas of skin unaffected by the skin condition and thus, any potential adverse effects to those areas of skin are unnecessary. Application of a treatment to those areas of skin unaffected by the skin condition is not expected be beneficial, which unnecessary wastes the treatment without providing any noticeable improvement in the aesthetic appearance or health of the skin.

### SUMMARY OF THE INVENTION

One exemplary embodiment of the present invention is directed to a handheld device for treating a skin condition. The device comprises an applicator arrangement applying a composition to skin. The composition comprising an active ingredient for treating the skin condition and a tracer. The device also comprises a detector arrangement obtaining image data corresponding to an image of an area of skin. The device further comprises a processing arrangement analyzing the image data to determine whether an artifact corresponding to the skin condition is detected at a location within the imaged area of the skin and to determine an amount of the tracer detected from the location. The processing arrangement directs the applicator arrangement to apply the composition to the location when the artifact is detected, and the amount of the tracer is less than a predetermined threshold level.

A method for treating a skin condition is also described. The method comprises obtaining, by a detector arrangement, image data corresponding to an image of an area of the skin. The method also comprises analyzing, by a processing arrangement, the image data to determine whether an artifact corresponding to the skin condition is detected at a location within the imaged area of the skin and to determine an amount of the tracer detected from the location. The method further comprises applying, by an applicator arrangement, a composition to the location when the artifact is detected from the location and the amount of the tracer is less than a predetermined threshold level. The composition comprises an active ingredient for treating the skin condition and the tracer.

These and other aspects of the invention will become apparent to those skilled in the art after a reading of the following detailed description of the invention, including the figures and appended claims.

### BRIEF DESCRIPTION OF THE FIGURES

FIG. 1a shows a block diagram of an exemplary device for treating a skin condition, according to an embodiment of the present application.

FIG. 1b shows the exemplary device of FIG. 1a in use for imaging an area of skin and applying a composition to the skin.

FIG. 2 shows an exemplary method for treating a skin condition, according to an embodiment of the present application.

FIG. 3 shows another exemplary method for treating a skin condition, according to an exemplary embodiment of the present application.

FIG. 4a shows a simulated image showing application of a cosmetic composition over multiple passes across a face of a user as described in Example III.

FIG. 4b shows a simulated image showing application of a skin brightening agent applied over multiple passes across a face of a user as described in Example III.

### DETAILED DESCRIPTION

The term “treat,” “treating,” or “treatment” as used herein refers to ameliorating, mitigating, preventing, improving, or eliminating the presence or signs of a condition or disorder.

The term “suitable for topical application” or “suitable for topical administration” as herein refers to those ingredients and/or treatments that are suitable for use on the skin, in particular, the skin of a human, without undue toxicity, incompatibility, instability, irritation, allergic response, unsightly visual appearance or the like.

The term “benefit agent” as used herein refers to any beneficial compound/composition/extract or active ingredient for treating a skin condition suitable for topical application. The skin condition, may include, for example, infection, inflammation, acne, uneven skin tone, sun damage, age spots, wrinkles, hyperpigmentation, eczema, hives, vitiligo, psoriasis, rosacea, warts, shingles, cold sore, uneven pigmentation and tone, redness/oxidative skin stress, in need of brightening, sagging/loss of elasticity, etc. Exemplary embodiments of benefit agents that may be incorporated into the composition are further described below.

A non-limiting list of useful benefit agents for acne includes benzoyl peroxide, retinoids including retinol, retinal, retinoic acid, retinyl acetate, and retinyl palmitate, hydroxy acids include, but are not limited, to glycolic acid, lactic acid, malic acid, salicylic acid, citric acid, and tartaric acid, sulfur, Zinc PCA (Zinc Pyrrolidone carboxylic acid), Allantoin (5-ureidohydantoin), Rosemary, 4-hexylresorcinol, N-acetyl glucosamine, gluconolactone, niacinamide, azelaic acid, and resveratrol.

A non-limiting list of useful pigmentation active benefit agents includes resorcinols, such as niacinamide, 4-hexyl resorcinol, curcuminoids (such as Sabiwhite (Tetrahydrocurcumin), phytic acid, resveratrol, soybean glycine soja oil, gluconolactone, azelaic acid, and retinoids including retinol, retinal, retinoic acid, retinyl acetate, and retinyl palmitate, enzymes such as laccase, tyrosinase inhibitors, melanin-degradation agents, melanosome transfer inhibiting agents including PAR-2 antagonists, exfoliants, sunscreens, retinoids, antioxidants, Tranexamic acid, tranexamic acid cetyl ester hydrochloride, skin bleaching agents, linoleic acid, adenosine monophosphate disodium salt, Chamomilla extract, allantoin, opacifiers, talcs and silicas, zinc salts, and the like. Examples of suitable tyrosinase inhibitors include but, are not limited to, Vitamin C and its derivatives, Vitamin E and its derivatives, Kojic Acid, Arbutin, resorcinols, hydroquinone, Flavones e.g., Licorice flavanoids, Licorice root extract, Mulberry root extract, Dioscorea Coposita root extract, Saxifraga extract and the like, Ellagic acid, Salicylates and derivatives, Glucosamine and derivatives, Fullerene, Hinokitiol, Dioic acid, Acetyl glucosamine, 5,5′-dipropyl-biphenyl-2,2′-diol (Magnolignan), 4-(4-hydroxyphenyl)-2-butanol (4-HPB), combinations of two or more thereof, and the like. Examples of vitamin C derivatives include, but are not limited to, ascorbic acid and salts, Ascorbic Acid-2-Glucoside, sodium ascorbyl phosphate, magnesium ascorbyl phosphate, and natural extract enriched in vitamin C. Examples of vitamin E derivatives include, but are not limited to, alpha-tocopherol, beta, tocopherol, gamma-tocopherol, delta-tocopherol, alpha-tocotrienol, beta-tocotrienol, gamma-tocotrienol, delta-tocotrienol and mixtures thereof, tocopherol acetate, tocopherol phosphate and natural extracts enriched in vitamin E derivatives. Examples of resorcinol derivatives include, but are not limited to, resorcinol, 4-substituted resorcinols like 4-alkylresorcinols such as 4-butyresorcinol (rucinol), 4-hexylresorcinol, phenylethyl resorcinol, 1-(2,4-dihydroxyphenyl)-3-(2,4-dimethoxy-3-methylphenyl)-Propane and the like and natural extracts enriched in resorcinols. Examples of salicylates include, but are not limited to, 4-methoxy potassium salicylate, salicylic acid, acetylsalicylic acid, 4-methoxysalicylic acid and their salts. In certain preferred embodiments, the tyrosinase inhibitors include a 4-substituted resorcinol, a vitamin C derivative, or a vitamin E derivative.

A non-limiting list of useful redness/antioxidant active benefit agents includes water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, propolis and extracts of feverfew. By “extracts of feverfew,” it is meant extracts of the plant “Tanacetum parthenium,” One particularly suitable feverfew extract is commercially available as about 20% active feverfew.

A non-limiting list of useful wrinkle active benefit agents includes N-acetyl glucosamine, 2-dimethylaminoethanol, copper salts such as copper chloride, peptides like argireline, syn-ake and those containing copper, coenzyme Q10, dill, blackberry, princess tree, Picia anomala, and chicory, resorcinols, such as 4-hexyl resorcinol, curcuminoids and retinoids including retinol, retinal, retinoic acid, retinyl acetate, and retinyl palmitate, hydroxy acids include, but are not limited, to glycolic acid, lactic acid, malic acid, salicylic acid, citric acid, and tartaric acid.

A non-limiting list of useful hydrating active benefit agents includes hyaluronic acid, and humectants. The hyaluronic acid may be linear, cross-linked, or a mixture of linear and cross-linked hyaluronic acid. It may be in a salt form, such as sodium hyaluronate. A humectant is a compound intended to increase the water content of the top layers of skin (e.g., hygroscopic compounds). Examples of suitable humectants include, but are not limited to, glycerin, sorbitol or trehalose or a salt or ester thereof.

A non-limiting list of useful brightening active benefit agents includes Vitamin C and its derivatives such as Ascorbic Acid 2-Glucoside, alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid.

A non-limiting list of useful benefit agents for sagging skin includes blackberry extracts, cotinus extracts, feverfew extracts, extracts of Phyllanthus niruri and bimetal complexes having copper and/or zinc constituents. The bimetal complex having copper and/or zinc constituents may be, for example, copper-zinc citrate, copper-zinc oxalate, copper-zinc tartarate, copper-zinc malate, copper-zinc succinate, copper-zinc malonate, copper-zinc maleate, copper-zinc aspartate, copper-zinc glutamate, copper-zinc glutarate, copper-zinc fumarate, copper-zinc glucarate, copper-zinc polyacrylic acid, copper-zinc adipate, copper-zinc pimelate, copper-zinc suberate, copper-zinc azealate, copper-zinc sebacate, copper-zinc dodecanoate, or combinations thereof.

Additional skin benefit agents or actives may include those actives listed in the following paragraphs. While some of these actives may have been listed above, they are included below to ensure a more robust listing.

Examples of suitable additional benefit agents include: skin lightening agents, darkening agents, anti-aging agents, tropoelastin promoters, collagen promoters, anti-acne agents, shine control agents, anti-microbial agents such as anti-yeast agents, anti-fungal, and anti-bacterial agents, anti-inflammatory agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, hydration boosters, efficacy boosters, anti-callous agents, agents for skin conditioning, anti-cellulite agents, fluorides, teeth whitening agents, anti-plaque agents, and plaque-dissolving agents, odor-control agents such as odor masking or pH-changing agents, and the like. Examples of various suitable additional cosmetically acceptable actives include UV filters such as but not limited to avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, carotenoids, free radical scavengers, spin traps, retinoids and retinoid precursors such as retinol, retinoic acid and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, amino acids such a proline, vitamins, lactobionic acid, acetyl-coenzyme A, niacin, riboflavin, thiamin, ribose, electron transporters such as NADH and FADH2, and other botanical extracts such as oat, aloe vera, Feverfew, Soy, Shiitake mushroom extracts, and derivatives and mixtures thereof.

Examples of suitable skin lightening benefit agents include, but are not limited to, tyrosinase inhibitors, melanin-degradation agents, melanosome transfer inhibiting agents including PAR-2 antagonists, exfoliants, sunscreens, retinoids, antioxidants, Tranexamic acid, tranexamic acid cetyl ester hydrochloride, skin bleaching agents, linoleic acid, adenosine monophosphate disodium salt, Chamomilla extract, allantoin, opacifiers, talcs and silicas, zinc salts, and the like.

Examples of suitable tyrosinase inhibitors include but, are not limited to, Vitamin C and its derivatives, Vitamin E and its derivatives, Kojic Acid, Arbutin, resorcinols, hydroquinone, Flavones e.g. Licorice flavanoids, Licorice root extract, Mulberry root extract, Dioscorea Coposita root extract, Saxifraga extract and the like, Ellagic acid, Salicylates and derivatives, Glucosamine and derivatives, Fullerene, Hinokitiol, Dioic acid, Acetyl glucosamine, 5,5′-dipropyl-biphenyl-2,2′-diol (Magnolignan), 4-(4-hydroxyphenyl)-2-butanol (4-HPB), combinations of two or more thereof, and the like. Examples of vitamin C derivatives include, but are not limited to, ascorbic acid and salts, Ascorbic Acid-2-Glucoside, sodium ascorbyl phosphate, magnesium ascorbyl phosphate, and natural extract enriched in vitamin C. Examples of vitamin E derivatives include, but are not limited to, alpha-tocopherol, beta, tocopherol, gamma-tocopherol, delta-tocopherol, alpha-tocotrienol, beta-tocotrienol, gamma-tocotrienol, delta-tocotrienol and mixtures thereof, tocopherol acetate, tocopherol phosphate and natural extracts enriched in vitamin E derivatives. Examples of resorcinol derivatives include, but are not limited to, resorcinol, 4-substituted resorcinols like 4-alkylresorcinols such as 4-butyresorcinol (rucinol), 4-hexylresorcinol (Synovea HR, Sytheon), phenylethyl resorcinol (Symwhite, Symrise), 1-(2,4-dihydroxyphenyl)-3-(2,4-dimethoxy-3-methylphenyl)-Propane (nivitol, Unigen) and the like and natural extracts enriched in resorcinols. Examples of salicylates include, but are not limited to, 4-methoxy potassium salicylate, salicylic acid, acetylsalicylic acid, 4-methoxysalicylic acid and their salts. In certain preferred embodiments, the tyrosinase inhibitors include a 4-substituted resorcinol, a vitamin C derivative, or a vitamin E derivative. In more preferred embodiments, the tyrosinase inhibitor comprises Phenylethyl resorcinol, 4-hexyl resorcinol, or ascorbyl-2-glucoside.

Examples of suitable melanin-degradation agents include, but are not limited to, peroxides and enzymes such as peroxidases and ligninases. In certain preferred embodiments, the melanin-inhibiting agents include a peroxide or a ligninase.

Examples of suitable melanosome transfer inhibiting agents including PAR-2 antagonists such as soy trypsin inhibitor or Bowman-Birk Inhibitor, Vitamin B3 and derivatives such as Niacinamide, Essential soy, Whole Soy, Soy extract. In certain preferred embodiments, the melanosome transfer inhibiting agents includes a soy extract or niacinamide.

Examples of exfoliants include, but are not limited to, alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid, and mechanical exfoliation such as microdermabrasion. In certain preferred embodiments, the exfoliant include glycolic acid or salicylic acid.

Examples of sunscreens include, but are not limited to, avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, and the like.

Examples of retinoids include, but are not limited to, retinol (Vitamin A alcohol), retinal (Vitamin A aldehyde), retinyl acetate, retinyl propionate, retinyl linoleate, retinoic acid, retinyl palmitate, isotretinoin, tazarotene, bexarotene, Adapalene, combinations of two or more thereof and the like. In certain preferred embodiments, the retinoid is selected from the group consisting of retinol, retinal, retinyl acetate, retinyl propionate, retinyl linoleate, and combinations of two or more thereof. In certain more preferred embodiments, the retinoid is retinol.

Examples of antioxidants include, but are not limited to, water-soluble antioxidants such as sulfhydryl compounds and their derivatives (e.g., sodium metabisulfite and N-acetyl-cysteine, glutathione), lipoic acid and dihydrolipoic acid, stilbenoids such as resveratrol and derivatives, lactoferrin, iron and copper chelators and ascorbic acid and ascorbic acid derivatives (e.g., ascobyl-2-glucoside, ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinones. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, black tea, white tea, pine bark, feverfew, parthenolide-free feverfew, oat extracts, blackberry extract, cotinus extract, soy extract, pomelo extract, wheat germ extract, Hesperedin, Grape extract, Portulaca extract, Licochalcone, chalcone, 2,2′-dihydroxy chalcone, Primula extract, propolis, and the like.

In some preferred embodiments, useful benefit agents for acne include, but are not limited, salicylic acid, Zinc PCA (Zinc Pyrrolidone carboxylic acid), Allantoin (5-ureidohydantoin), Rosemary, 4-hexylresorcinol, N-acetyl glucosamine, gluconolactone, niacinamide, azelaic acid, and resveratrol.

In some preferred embodiments, a list of useful pigmentation active benefit agents includes tetrahydrocurcumin, phytic acid, resveratrol, soybean glycine soja oil, gluconolactone, laccase, 4-hexyl resorcinol, N-acetyl glucosamine, gluconolactone, niacinamide, azelaic acid, and resveratrol.

In some preferred embodiments, a list of useful active benefit agents includes to simultaneously treat acne and pigmentation includes 4-hexyl resorcinol, N-acetyl glucosamine, gluconolactone, niacinamide, azelaic acid, and resveratrol.

The term “frexel” as used herein refers to a small pixel-like region of skin, which corresponds to a single large pixel or a small number of pixels in a digitally obtained image. For example, a frexel may correspond to a skin area having an average diameter from about 1/15 to about ⅕ inch.

The present application provides a device and method for treating a condition of the treatment surface and applying a tracer to track a level of treatment previously administered to the treatment surface. The treatment may comprise administering a composition having a benefit agent for treating the condition or may comprise administering a therapy without a benefit agent that can otherwise impart a beneficial effect to the skin (e.g., light therapy and/or laser treatment). It is contemplated that the treatment may be applied to any suitable treatment surface, such as, an interface between a biological surface and the external environment (e.g., air), in particular, a topical surface. Suitable biological surfaces may include keratinous surfaces (such as, but not limited to, surfaces of the skin, hair, and/or nails), and enameled surfaces (e.g., a surface of a tooth). Preferably, the treatment surface is that of a mammal or a human. Although exemplary embodiments are discussed herein relating to the skin, it is contemplated that the device and method of the present application may be used to selectively apply a treatment and a tracer to any suitable treatment surface.

More particularly, the present application provides a device and method for selectively administering a treatment to the skin (e.g., of a mammalian or human face) for treating a skin condition (e.g., excessive melanin deposits, infection, inflammation, acne, wrinkles, and nonuniformities in skin color). The device and method identify skin artifacts (e.g., redness from infection and/or inflammation, acne, erythema, pre-emergent acne, uneven skin tone, sun damage, age spots, wrinkles, etc.) and control administration of a treatment for a skin condition associated with the skin artifacts to reduce appearance of the artifacts, and improve skin health and/or over-all aesthetic appearance of the skin. The device of the present application analyzes an image of an area of skin to identify locations to which the composition should be applied, e.g., locations at which skin artifacts are detected, and to determine amounts of tracer detected from the identified locations. The device controls treatment of the skin artifacts based on the amounts of tracer detected. As will be explained further below, the device includes a detector arrangement for collecting data corresponding to an area of skin. The data is further analyzed to detect a skin condition and/or quantify an amount of tracer previously deposited onto the area of skin. The device analyzes the data to determine whether an artifact corresponding to the skin condition is detected and applies a treatment for the skin condition to the areas of skin based on this analysis. The analysis may generate an optimum level of a treatment for treating the skin condition. The device further determines an amount of treatment previously administered to the area of skin based on the amount of tracer detected from the area of skin and applies further treatment to the when the amount of tracer indicates that less than the optimum level of treatment has been previously applied to the area of skin.

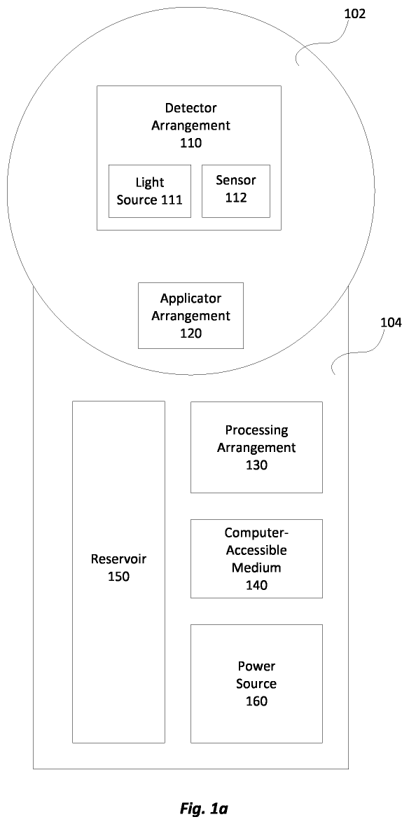

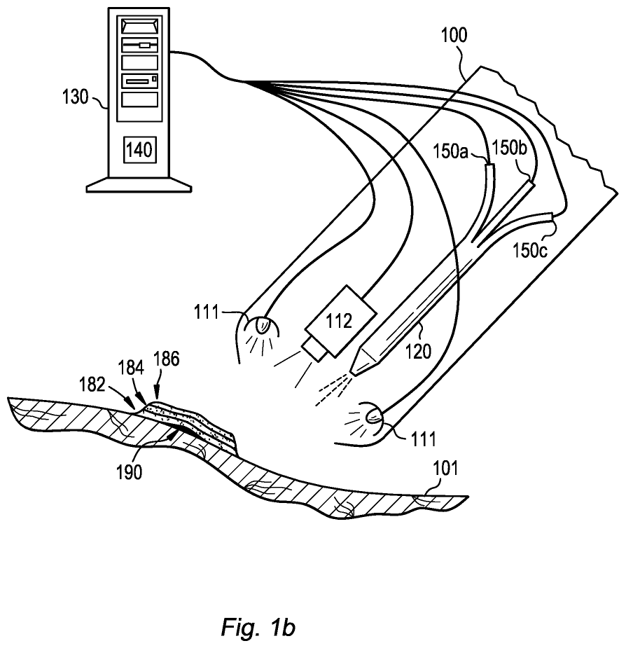

FIGS. 1a and 1b show an exemplary device 100 for treating a skin condition by applying a topical composition to the skin. FIG. 1b further shows the exemplary device 100 in use for applying a benefit agent 182, a tracer 184, and/or a cosmetic composition 186 to an artifact 190 to the skin 101. Although the benefit agent 182, the tracer 184 and the cosmetic composition 186 are shown in FIG. 1b in three separate layers, it is contemplated that they may be mix together in any combination and may be applied in any order onto the skin 101. The device 100 of this embodiment is sized and shaped to be a handheld device designed to be held within a palm of a user's hand. The device 100 according to this embodiment comprises a head portion 102 and a handle portion 104. The handle portion 104 of the device 100 has an elongated shape defining a cavity for housing components therein. In some embodiments, the handle portion 104 is sized and shaped to be held within the palm of the user's hand. In other embodiments, the handle portion 104 is sized and shaped to be held by the fingertips of the user's hand.

The head portion 102 of the device 100 according to this embodiment comprises a detector arrangement 110 obtaining image data corresponding to an image of an area of skin. The head portion 102 of this embodiment also comprises an applicator arrangement 120 selectively applying a composition to portions of the skin as directed by a processing arrangement 130 based on image data from the detector arrangement 110. In some embodiments, the detector arrangement 110 and the applicator arrangement 120 are part of an inset portion 106 of the head portion 102 such that when the head portion 102 is placed over an area of skin to be treated, the inset portion 106 is not in contact with the skin.

The detector arrangement 110 comprises at least one light source 111 delivering light (e.g., visible, infra-red light, red light, blue light, green light and/or ultra-violet light) to an area of skin, and at least one sensor 122 detecting light reflected from the area of the skin. The light source(s) 111 may comprise any suitable light emitting device for illuminating the area of skin with visible, infra-red light, red light, blue light, green light and/or ultra-violet light. For example, the light source(s) 111 may comprise one or more LEDs. The light source(s) 111 are selected and arranged to provide an amount of illumination over the area of skin sufficient to detect and/or measure reflectance of light by the skin. Furthermore, at least one light source 111 is selected and arranged to provide an amount of illumination over the area of skin for detecting and/or measuring an amount of a tracer, as will be discussed further below, on the skin. Preferably, the light source(s) 111, collectively, provide a substantially uniform distribution of light over the area of skin being imaged.

In one embodiment, the light source 111 comprises a white color LED. The white color LED may provide composite light having a least two separate peaks of radiant power at different wavelengths, corresponding to two different bands of wavelengths of light. Specifically, the white LEDs provide peak radiant power at two different wavelengths. For example, the white LEDs may have a color temperature ranging from 3000 K to 5700 K and/or a Color Rendering Index (CRI) of 70 to 80. In specific embodiments, the white LEDs may have a color temperature of 5700 K and/or CRI of 70; a color temperature of 4000 K and/or CRI of 75; or a color temperature of 3000 K and/or CRI of 80. Preferably, the white color LED may have a color tone ranging from a cool color temperature to a warm color temperature (having a first peak/band within a blue spectrum and a second peak/band within a green spectrum).

The light source 111 may be selected to provide a peak radiant power at a desired wavelength for detecting and/or measuring an amount of a tracer on the skin. In other embodiments, the detector arrangement 100 comprises a plurality of light sources 111 each having a different peak wavelength. The wavelength at which each light source provides a peak intensity (e.g., a peak radiant power) is referred to herein as a peak wavelength. For example, each of the light sources 111 may emit a different one of a red light, a blue light, a green light, an infra-red light, and an ultra-violet light.

In one embodiment, the detector arrangement 110 comprises a first light source for delivering a red or far-red light. The detector arrangement 110 may further comprise a second light source for delivering a green light and/or a third light source for delivering a blue light or an infra-red light. Specifically, the detector arrangement 110 may comprise three light sources 111 each delivering a light having peak wavelengths at or about the wavelengths specified below in Table 1.

TABLE 1

| First Light Source | Second Light Source | Third Light Source |
|---|---|---|
| 450 nm | 540 nm | 610 nm |
| 450 nm | 540 nm | 690 nm |
| 840 nm | 540 nm | 610 nm |
| 840 nm | 540 nm | 690 nm |

The sensor(s) 112 may comprise any suitable components for detecting light from the skin. For example, the sensor 112 may be sensitive to an amount of reflected light and/or emitted light in one or more wavelengths from the skin. Furthermore, at least one sensor 112 is selected to detect and/or measure an amount of a tracer present on the skin. Suitable sensors 112 may include, for example, optical sensors, photographic or video cameras, photodiodes and/or phototransistors as would be understood by those skilled in the art. The sensor 112 of the detector arrangement 120 may be an RGB camera which can detect light in red, green and/or blue channels of the sensor 112. In one embodiment, the sensor 112 may be an optical camera having one or more filter arrays for detecting components of light received within different wavelength ranges. For example, the sensor 112 may be an optical camera having a color filter array (e.g., a Bayer filter array) to separately detect each of a red, a green and a blue color component of light. For example, the optical camera may have at least three different color filters having different levels of sensitivity (e.g., in % quantum efficiency) across the visible spectrum. The color filter array may include (1) a red color filter (e.g., having a peak sensitivity at or about 610 nm wavelength); (2) a green color filter (e.g., having a peak sensitivity at or about 540 nm wavelength); and (3) a blue color filter (e.g., having a peak sensitivity at or about 450 nm wavelength). Each of these color filters provides peak sensitivity at different wavelengths. The sensor 112 may be selected to provide a peak sensitivity at a desired wavelength for detecting and/or measuring an amount of a tracer on the skin.

The detector arrangement 110, including the light source(s) 111 and sensor(s) 112, is operably connected to a processing arrangement 130 to execute instructions stored on a computer-accessible medium 140. The processing arrangement 130 in this embodiment controls the light source(s) 111 and receives and analyzes imaging data received from the sensor(s) 112.

It is contemplated that the processing arrangement 130 and the computer-accessible medium 140 may be positioned anywhere within or external to the device 100. In one embodiment, as shown in FIG. 1a, the processing arrangement 130 and the computer-accessible medium 140 are located within the handle portion 104. Alternatively, as shown in FIG. 1b, the processing arrangement 130 and the computer-accessible medium 140 are located external to the device 100 and may be operably connected to the device 100 via a wired or wireless connection. The processing arrangement 130 in this embodiment also controls the applicator arrangement 120 to selectively apply the composition to desired frexels. The processing arrangement 130 may be, e.g., entirely or a part of, or include, but is not limited to, a computer/processor that can include, e.g., one or more microprocessors, and use instructions stored on a computer-accessible medium 140 (e.g., memory storage device). The computer-accessible medium 140 may, for example, be a non-transitory computer-accessible medium containing executable instructions therein. A storage arrangement may be provided separately from the computer-accessible medium 140, which may provide the instructions to the processing arrangement 130 to configure the processing arrangement 130 to execute certain exemplary procedures, processes and methods.

The applicator arrangement 120 according to this embodiment comprises a suitable composition application device for depositing a topical composition comprising a benefit agent and a tracer onto frexels. An exemplary topical composition application device in this embodiment includes, for example, a sprayer (e.g., an electronic sprayer or airbrush sprayer), a drop control device, or any other suitable application device for applying a composition in small drops to desired locations as would be understood by those skilled in the art. In one exemplary embodiment, the applicator arrangement 120 comprises a nozzle for depositing a pressurized liquid or viscous composition. The nozzle may be any suitable device for depositing a thin layer of a composition onto frexels. For example, the nozzle may comprise a first chamber holding the liquid or viscous composition and a second chamber containing a propellant to be mixed with the composition when the composition is dispensed to a desired location. Although an exemplary embodiment of the nozzle is described above, it is contemplated that the device of the present application may include any suitable nozzle for dispensing droplets of a composition under pressure as would be understood by those skilled in the art.

The applicator arrangement 120 is operably connected to a reservoir 150 containing a topical composition to be applied to the skin, such that the composition within the reservoir 150 can be transferred from the reservoir 150 to the applicator arrangement 120 for deposition onto the skin. The topical composition comprises a benefit agent and/or a tracer. The topical composition may further include cosmetic ingredients for modifying an appearance of the skin, ingredients for imparting a further benefit to the skin, such as a moisturizer for hydration, or a carrier. In one embodiment, the reservoir 150 contains a composition comprising a benefit agent and a tracer. Alternatively, the applicator arrangement 120 is operably connected to two separate reservoir(s) 150: a first of which containing a composition comprising the benefit agent, and a second of which containing a composition comprising a tracer. The applicator arrangement 120 may be configured to transfer these two compositions in a predetermined ratio to form a mixture and apply said mixture onto the skin.

In another embodiment, the applicator arrangement 120 is operably connected to a plurality of reservoir(s) 150 each containing a different composition therein. The reservoir(s) 150 may contain compositions having a benefit agent, a tracer, cosmetic ingredient(s), or combinations thereof, each of these components are discussed in further detail below. In one example, the applicator arrangement 120 is operably connected to two reservoirs 150: a first reservoir containing a composition comprising a benefit agent and a tracer, and a second reservoir containing a cosmetic composition. The cosmetic composition may include any suitable cosmetic ingredient(s) for modifying an appearance of the skin. The compositions of the first and second reservoirs may further include ingredients for imparting a further benefit to the skin, such as a moisturizer for hydration, or a carrier. In this example, the applicator arrangement 120 may be configured to separately transfer and apply onto the skin (e.g., as separate pulses of each composition dispensed by the applicator arrangement 120) the composition of the first reservoir and the cosmetic composition of the second reservoir. In addition or alternatively, the applicator arrangement 120 may be configured to transfer the two compositions from the first and second reservoirs in amounts specified by the processing arrangement 130 to form a mixture, and apply said mixture onto the skin. In another example, as shown in FIG. 1b, the applicator arrangement 120 is operably connected to a first reservoir 150a containing a composition comprising a benefit agent, a second reservoir 150b containing a composition comprising a tracer, and a third reservoir 150c containing a cosmetic composition. In this embodiment, the applicator arrangement 120 is configured to transfer any one or more of the compositions of the first, second and third reservoir(s) 150a, 150b, 150c in amounts specified by the processing arrangement 130 for application on to the skin.

The applicator arrangement 120 is fluidly connected by a series of conduits, valves, and/or pressure sources to the reservoir(s) 150. It is contemplated that the reservoir(s) 150 may be housed anywhere within the device 100. In one exemplary embodiment, as shown in FIG. 1a, the reservoir(s) 150 are housed within the handle portion 104 of the device 100. In some embodiments, the reservoir(s) 150 may be removeable container(s) that can be replaced upon exhaustion of the contents therein. For example, the reservoir(s) 150 may be pressurized canister(s) containing composition(s) to be applied to the skin therein.

The tracer may be any compound, composition, or ingredient suitable for topical application to the skin. As will be discussed further below, the tracer is applied by the applicator arrangement 120 in an amount proportional to a level of treatment administered to the skin. For example, the tracer may be applied by the applicator arrangement 120 in an amount proportional to the amount of benefit agent applied to the skin so that the amount of tracer detected from a frexel on the skin can be correlated to an amount of benefit agent previously administered to the frexel. The tracer may be a dye or pigment suitable for topical application. The tracer may be selected to absorb and/or emit a light having a peak wavelength that is distinct from light absorbed by naturally occurring coloring of an artifact on the skin, such as, for example, eumelanin, oxyhemoglobin, deoxyhemoglobin, skin coloration, etc., so that the tracer is separately detectable from naturally occurring coloring of the skin by the detector arrangement 110.

In one example, the tracer may be a compound, composition or ingredient that suppresses detection of a skin artifact. For example, such a suppression tracer may be, for example, a color cosmetic applied to the skin artifact such that image data subsequently obtained from an area of skin containing the skin artifact is suppressed from detecting the skin artifact (e.g., a skin discoloration being mitigated by color cosmetic). The suppression tracer may be administered in an amount proportional to an artifact magnitude of the skin artifact. In another example, the tracer may be a compound, composition or ingredient that minimally interferes with detection of a skin artifact and therefore, allows for application of an active (and its tracer) independent of an artifact magnitude of the skin artifact. This is particularly useful in controlling administration of a benefit agent, where the benefit agent needs to be administered above a minimum therapeutic threshold to impart beneficial effects, while not being applied in excess to cause detrimental effects (e.g., toxicity).

In some embodiments, the tracer is a dye or a pigment that is visible to the human eye, for example, the tracer is absorbs a light having a wavelength within the visible light spectrum (e.g., from about 400 nm to about 700 nm). For example, the tracer may be a dye or a pigment that absorbs red light (e.g., light having a peak wavelength from about 600 nm to about 700 nm), green light (e.g., light having a peak wavelength from about 500 nm to about 565 nm), or blue light (e.g., light having a peak wavelength from about 450 nm to about 485 nm). In particular, the tracer absorbs light having a peak wavelength corresponding to the strongest intensity for a primary color. For example, the tracer absorbs light having a peak wavelength at or about 610 nm (i.e., peak wavelength of red observed by a standard observer), at or about 540 nm (i.e., peak wavelength of green observed by the standard observer), or 450 nm (i.e., peak wavelength of blue observed by the standard observer). In another example, the tracer absorbs red light having a peak wavelength from about 640 nm to about 650 nm. The peak wavelengths for a standard observer as discussed above is based on the CIE 1931 standard observer color-mapping function. Because the tracer of this embodiment is visible when applied to the skin, the tracer may be administered as a cosmetic ingredient in a cosmetic composition that is applied to reduce appearance of artifacts and/or enhance the aesthetic appearance of skin.

In another embodiment, the tracer may be a dye or a pigment that is weakly visible such that it appears faint to the human eye. The tracer may be a dye or a pigment having a peak wavelength that is substantially higher or substantially lower than the wavelengths at peak wavelength observed by a standard observer for each of the primary colors. Preferably, the tracer absorbs a narrow band of light corresponding to peak sensitivity of at least one sensor 112 of the device 100. Exemplary tracers may include dyes and/or pigments having a weakly visible cyan color absorbing light in a far-red spectrum (e.g., having a peak wavelength from about 650 nm to about 700 nm). More specifically, the tracer absorbs a red light having a peak wavelength at or about 690 nm but does not significantly absorb light having a peak wavelength at or about 610 nm. Other tracers may include dyes and/or pigments having a weakly visible magenta color absorbing green light having a peak wavelength from about 500 nm to about 600 nm and dyes and/or pigments having a weakly visible yellow color absorbing blue light having a peak wavelength from about 400 nm to about 500 nm. Suitable visible or weakly visible tracers may include sulfonated or non-sulfonated cyanine dyes. For example, the tracer is a non-sulfonated cyanine dye absorbing light having peak wavelengths from about 550 nm to about 700 nm, such as those commercially available from Lumiprobe Corporation.

In another embodiment, the tracer is not visible to the human eye. The tracer may be a fluorescent dye and/or pigment that is invisible to the human eye under ambient light conditions but fluoresces when excited by an energy source (e.g., light). The fluorescent tracer may be excited by at least one light source 111 of the detector arrangement 110 delivering a visible light, an infra-red light (e.g., having a wavelength from about 700 nm to about 1 mm), or an ultra-violet light (e.g., having a wavelength from about 10 nm to about 400 nm). When the light is delivered to the fluorescent tracer, photons from the light are absorbed by electrons of the tracer to transition from a grounded state to an excited state at higher valence level. As the electrons return to the grounded state from the excited state, energy is released in the form of a light emission that can be detected by at least one sensor 112 of the detector arrangement 110. For example, the fluorescent tracer may comprise fluorochromes, such as those commercially available under the tradename Chromis from Cyanagen or CF® Dyes from Biotium. Suitable fluorescent tracers may include, for example, dipyrrometheneboron difluorides (BDPs), trimethine cyanines, pentamethine cyanines, eptamethine cyanines, and coumarines. For example, the fluorescent tracers may include fluorescent dyes having peak emissions wavelengths from about 400 nm to about 850 nm.

The cosmetic ingredients may include any suitable ingredients for topical application to the skin for modifying an appearance of the skin, such as, for example, an opaque substance, a tinted cosmetic, or any other suitable compositions for enhancing the appearance of skin. In one embodiment, the cosmetic ingredients comprise reflectance modifying agents (RMAs) (any component useful for altering reflectance of the skin). For example, suitable RMAs may include inks, dyes, pigments, bleaching agents, chemically altering agents and other substances that may be used to alter the reflectance of the skin. Some suitable RMAs may include a transparent RMA, such as a dye or a diluted pigment. Other suitable RMAs may include an opaque RMA having high refractive index particles. In particular, the high refractive index particles may comprise particles having a refractive index of 2.0 or greater. In one specific example, the RMA may comprise particles of titanium dioxide. Specifically, the titanium dioxide particles may be uniformly distributed and/or suspended in the cosmetic composition.

In some embodiments, the head portion 102 may also optionally include a treatment arrangement (not shown) for administering a therapy without a benefit agent that can otherwise treat a skin condition or impart a beneficial effect to the skin. For example, the treatment arrangement may provide light therapy or laser treatment to lighten, reduce pigmentation, reduce inflammation, reduce infection to a frexel on the skin. In this embodiment, the tracer may be applied by the applicator arrangement 120 in an amount proportional to a level of intensity of treatment applied by the treatment arrangement to the skin so that the amount of tracer detected from a frexel on the skin can be correlated to an intensity of treatment previously administered to the frexel.

The device 100 according to this embodiment further comprises a power source 160 providing power to control and operate the device 100. It is contemplated that the power source 160 may be located anywhere within the device 100 or may alternatively be external to the device 100. In one exemplary embodiment, as shown in FIG. 1, the power source 160 which is housed within the handle portion 104 of the device 100, is operably connected to the detector arrangement 120, the applicator arrangement 130 and/or the processing arrangement 130. Those skilled in the art will understand that various known suitable sources of power may be used. For example, the power source 10 may comprise a battery or a connection to an external source of power. In particular, the power source 10 may comprise a rechargeable battery device.

In use, the head portion 102 is placed over an area of skin to be treated. During use, the device 100 may be utilized to image a plurality of different areas of skin. For example, the head portion 102 may be moved across a surface of the skin allowing the device 100 to continuously image (at any desired frame rate) different areas of the skin to obtain image data and analyze the image data to selectively administer a treatment for treating a skin condition and apply a composition comprising a tracer at desired frexels (locations on the skin). More particularly, during a use session, the user may move the head portion 102 back and forth across the surface of the skin in multiple passes to allow the device 100 to review previously treated areas to detect artifacts which were missed or incompletely addressed and further treat identified artifacts on the skin.

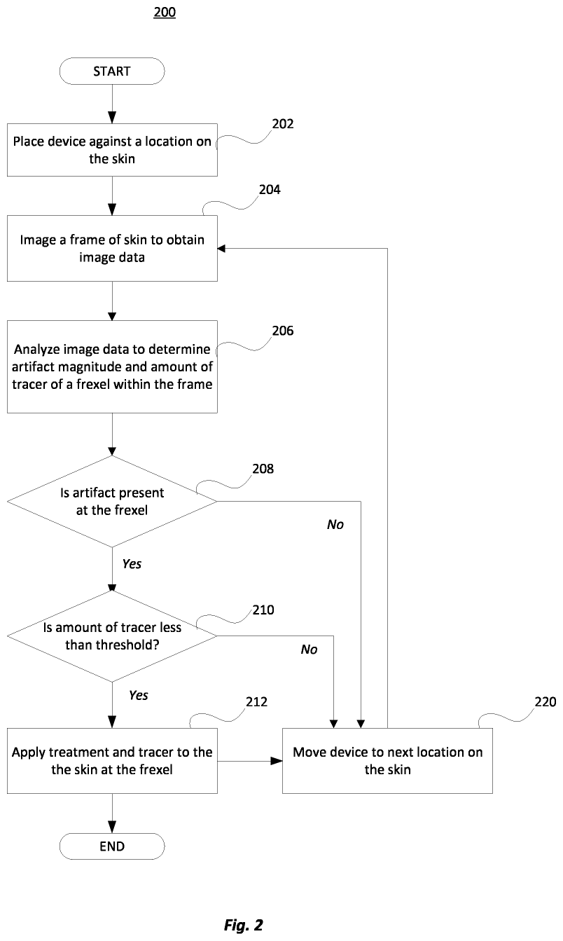

The present application also includes a method for treating a skin condition. An exemplary method 200 is shown in FIG. 2. In step 202, the user may initiate use of the device 100 by placing a head portion 102 of the device 100 against a surface of skin, for example, the skin of the face. The head portion 102 covers an area of skin, e.g., an area constituting a frame to be imaged and analyzed by the device 100.

In step 204, the detector arrangement 110 images the area to obtain image data for the area of the skin. To image the area of skin, the detector arrangement 110 illuminates the area of skin using the light source(s) 111 and records an image for generating the image data with the sensor(s) 112. In an exemplary embodiment, at least one of the light source(s) 111 illuminating the area of skin may have a peak wavelength suitable for detecting an underlying biologic component of a skin artifact (e.g., redness from infection and/or inflammation, acne, erythema, pre-emergent acne, uneven skin tone, sun damage, age spots, wrinkles, etc.) in need of treatment. More particularly, the light source(s) 111 illuminating the area of skin may have at least one peak wavelength corresponding to a peak absorption wavelength of the underlying biologic component, such as for example, eumelanin, oxyhemoglobin, deoxyhemoglobin, skin coloration, etc.

In another embodiment, step 204 utilizes a plurality of different light sources 111 each illuminating the area of skin with a light having a different wavelength (or the light sources are arranged such that they impinge on the skin from different directions). The light sources 111 in this embodiment may have wavelengths suitable for providing differential detection of an underlying biologic component of a skin artifact. In particular, two of the plurality of light sources 111 in this embodiment may have different peak wavelengths such that a comparison of image data acquired under illumination with at least two of these different light sources 111 can be used to detect and/or measure an amount of an underlying biologic component of a skin artifact. For example, step 204 illuminates the area of skin with three differently colored lights: (1) a red or far-red light, (2) a green light and (3) a blue light or an infra-red light. Specifically, step 204 may illuminate the area of skin with lights having peak wavelengths at or about the wavelengths specified above in Table 1. Each of the plurality of light sources 111 may be provided concurrently to illuminate the area of skin for recording an image with the sensor(s) 112. Alternatively, each of the plurality of light sources 111 may be provided sequentially to illuminate the area of skin for recording a plurality of separate images, each image illuminated with each corresponding light source 111.

In step 206, the processing arrangement 130 analyzes the image data from the detector arrangement 110 to determine whether an artifact corresponding to a skin condition is detected at a frexel within the imaged area of the skin and to determine an amount of the tracer detected from the frexel. The processing arrangement 130 may analyze the image data by any suitable methods to determine whether an artifact is present at a frexel in the imaged area. In one example, the processing arrangement 130 analyzes the image data to determine an artifact magnitude of the frexel, and determine that an artifact is indeed detected at the frexel when the artifact magnitude is greater than a predetermined threshold level. The artifact magnitude corresponds to an intensity of appearance of the skin artifact (e.g., intensity of redness of skin, darkness of skin discoloration, intensity of appearance of wrinkles). The processing arrangement 130 also analyzes the image data to determine an amount of the tracer, if any, is detected from the frexel. As shown in step 208, if an artifact is detected at the frexel, then the method 200 proceeds to step 210. If an artifact is not detected, then the frexel of skin is not detected by the device 100 as in need of treatment. Therefore, the method 200 does not apply any treatment or composition to the frexel and the method 200 proceeds to step 220.

In step 210, the method 200 compares whether the amount of tracer detected from the frexel is less than a predetermined threshold level. If the amount of tracer detected is less than the predetermined threshold level, then the method 200 proceeds to step 212. If the amount of tracer detected is at or above the predetermined threshold level, then the method 200 proceeds to step 220. As will be discussed further below, the tracer is applied by the applicator arrangement 120 in an amount proportional to a level of treatment administered to the skin. Therefore, the amount of tracer detected corresponds to a level of treatment previously administered to the skin. The method 200 utilizes the amount of tracer detected to track the level of treatment previously administered to the skin and control a total level of treatment administered to each frexel where an artifact is detected over the course of a use session. In particular, the amount of tracer detected corresponds to an amount of benefit agent previously administered to the frexel. As the head portion 102 is moved back and forth in multiple passes, a frexel may be detected more than once. The amount of tracer detected at the frexel corresponds to a total amount of benefit agent accumulated on the frexel from the multiple passes in a use session. The predetermined threshold level is selected to control a maximum total amount of benefit agent that can be applied to the frexel in a use session.

Effect of a benefit agent generally imparts a therapeutic benefit along a dose response curve indicating that as the dosage of the benefit agent increases, the therapeutic response to the benefit agent increases until it approaches a plateau at higher doses. The benefit agent may also cause adverse effects. Typically, the adverse effects depend on dosage along an adverse effect curve where the adverse effect of the benefit agent at low doses is minimal or low but increases as the dosage increases. Such adverse effects may include undue toxicity, incompatibility, instability, irritation, allergic response, unsightly visual appearance or the like. The predetermined threshold level for the tracer may correspond to a desired dosage level threshold for the benefit agent selected to correspond to a dosage that is titrated to balance therapeutic benefits against adverse effects of the benefit agent. In some embodiments, the desired dosage level threshold may correspond to a maximum dose that can be safely administered to the skin of the user. In other embodiments, the desired dosage level threshold may correspond to a dose sufficient to impart therapeutic effects while avoiding undue visible irritation and/or discoloration to the skin.

In step 212, the processing arrangement 130 directs the applicator arrangement 120 to administer a treatment for a skin condition to the frexel. In particular, the treatment is application of a topical composition comprising a benefit agent for treating the skin condition. In one embodiment, the applicator arrangement 120 applies a pulse of a fixed dose of a composition comprising the benefit agent and the tracer. The tracer is part of the same composition as the benefit agent and is therefore, applied in a fixed amount proportional to the amount of benefit agent in each fixed dose of the composition. Alternatively, the applicator arrangement 130 may be configured to apply two separate pulses sequentially: (1) a first fixed dose of a first composition comprising the benefit agent; and (2) a second fixed dose of a second composition comprising the tracer. The second composition is preferably applied concurrently with, or within a close time frame to, the first composition so that a total amount of the second composition accumulated on a frexel is maintained to be proportional to a total amount of the first composition, as the head portion is moved back and forth in multiple passes in a use session.

In another embodiment, the applicator arrangement 120 applies a pulse having a variable dose of a composition comprising the benefit agent and the tracer. The variable dose is determined by the processing arrangement 130 based on the artifact magnitude of a frexel and the amount of the tracer detected from the frexel. In an exemplary embodiment, the process arrangement 130 determines a total desired dose of the composition as a function of the artifact magnitude. For example, the processing arrangement 130 may analyze an artifact magnitude (e.g., intensity of redness for an erythema, intensity of a dark spot) at a frexel and determine a total amount of the benefit agent for treating the artifact at the frexel. The total amount of the benefit agent is a therapeutically effective amount of the benefit agent for treating a skin condition corresponding to the artifact. In addition, the processing arrangement 130 determines an accumulated amount of the benefit agent previously applied to the frexel, based on the amount of tracer detected from the frexel. The processing arrangement 130 then determines the variable dose to be applied by the applicator arrangement 120 as a difference of the total amount of the benefit agent for treating the artifact and the accumulated amount of the benefit agent previously applied to the frexel. Alternatively, the applicator arrangement 130 may be configured to apply two separate pulses sequentially: (1) a first variable dose of a first composition comprising the benefit agent; and (2) a second variable dose of a second composition comprising the tracer. The processing arrangement 130 determines the first variable dose of the first composition in the manner described above and determines the second variable dose as having a value proportional to the first variable dose. Similar to the fixed dosed embodiments described above, the second composition is preferably applied concurrently with, or within a close time frame to, the first composition so that a total amount of the second composition accumulated on a frexel is maintained proportional to a total amount of the first composition, as the head portion is moved back and forth in multiple passes in a use session.

In step 220, the device 100 is moved by the user to a new frame or area of the skin and the process is repeated. This movement may be detected by the device 100 by any suitable means, such as, for example, an accelerometer or by image analysis. The method 200 then returns to step 204 and images, analyzes and selectively applies treatment for a skin condition and a tracer to track an amount or level of treatment previously administered to the skin, as determined by the device 100, to this new area of skin in the same manner described above. It is noted that the method 200 may be interrupted and terminated by the user before any one of steps 204 through 212 by any suitable operation, such as, for example, removing the device 100 from the skin or switching off the device 100, in particular, the power source 160 of the device.

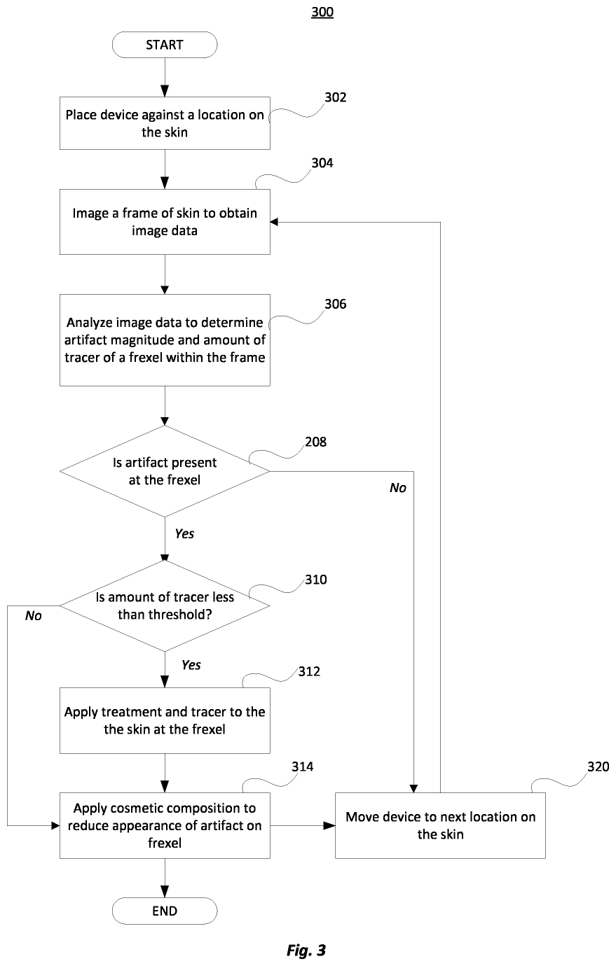

Another exemplary method 300 is shown in FIG. 3. Steps 302 to 308, step 312 and step 210 are the same as steps 202 to 208, step 212 and step 220 as discussed above with respect to method 200, respectively. Step 310 is substantially similar to step 210, except as noted in FIG. 3 and discussed below. In step 310, the method compares whether the amount of tracer detected from the frexel is less than a predetermined threshold level. If the amount of tracer detected is at or above the predetermined threshold, then the method 300 proceeds to step 314. Step 310 limits the total level of treatment administered to each frexel over the course of a use session, but proceeds in a separate step to further apply a cosmetic application when an artifact is detected at a frexel. In step 314, the processing arrangement 130 directs the applicator arrangement 120 to apply a cosmetic composition to the frexel. The amount of the cosmetic composition applied by the applicator arrangement 120 may be a fixed amount or may be variably selected by the processing arrangement 130 as a function of the artifact magnitude determined in step 306. Furthermore, the variable amount of the cosmetic composition applied in step 314 may be determined independently from the level of treatment for the skin condition administered in step 312. Although steps 312 and 314 of method 300 are shown sequentially in FIG. 3, steps 312 and 314 may be performed in reverse order (i.e., if step 310 is yes, then proceed to step 314 and then step 312). Alternatively, steps 312 and 314 may be performed independently (i.e., if step 310 is yes, then proceed to each of steps 312 and 314 independently). For example, step 312 may be performed by one or more nozzles for applying the treatment and tracer to the skin whereas step 312 may be performed by a separate nozzle for applying the cosmetic composition to the skin.

In an exemplary embodiment, the processing arrangement 130 analyzes the image data to determine whether pre-emergent acne may be present. Specifically, the processing arrangement 130 analyzes the image data to detect presence of reddening on the skin, which is believed to correspond to an increased level of hemoglobin at the area of skin. The processing arrangement 130 may analyze the image data to separate spectral contributions from hemoglobin from other skin components, such as, for example, melanin. In particular, the processing arrangement 130 may analyze the image data and compare the image data to normative data to generate normalized values for spectral contributions from hemoglobin without normative spectral contributions from other skin components. In particular, the image data may be obtained by sensor(s) 122 that provide red, green and blue reflectance measurements. Each of these reflectance measurements may be represented in the image data as a vector and compared to a lookup table containing normative data corresponding to empirically generated estimates for amount of hemoglobin present in the skin independent of spectral contributions from other skin components. The processing arrangement 130 may determine an amount of hemoglobin detected based on the image data and compare the amount to a proximal level of hemoglobin in the area around the imaged area of skin and generate a normative background value for blushing and other effects that cause skin perfusion in the nearby region. If the processing arrangement 130 determines that a pre-emergent acne lesion is detected, the device 100 may administer a treatment, in particular, a therapeutically active agent, e.g., salicylic acid, to reduce the appearance of or prevent eruption of the pre-emergent acne. The amount or level of treatment administered may be determined as a function of a normalized amount of hemoglobin detected from the area of skin. In some embodiments, a fixed optimum amount or level of treatment may be desired when the normalized amount of hemoglobin detected is at or above a predetermined threshold. In other embodiments, a variable optimum amount or level of treatment determined as a function of the normalized amount of hemoglobin detected may be desired when it is at or above the predetermined threshold. For example, a high normalized amount of hemoglobin detected may correspond to an erupted acne and therefore a lesser dosage of a therapeutically active agent, e.g., salicylic acid, may be desired to not further irritate skin at the erupted lesion. The processing arrangement 130 may further analyze the image data to determine an amount of tracer detected and correlate the amount of tracer to an amount or level of treatment previously applied to the area of skin. If the amount or level of treatment previously applied is less than the optimum amount of level, as determined above, then further treatment is applied by the device 100. The amount or level of the further treatment may be fixed or may be a variable amount determined as difference between the optimum amount or level of treatment, as determined above, and the amount or level of treatment previously applied to the area of skin.

Those skilled in the art will understand that the exemplary embodiments described herein may be implemented in any number of manners, including as a separate software module, as a combination of hardware and software, etc. For example, the exemplary methods may be embodiment in one or more programs stored in a non-transitory storage medium and containing lines of code that, when compiled, may be executed by one or more processor cores or a separate processor. A system according to one embodiment comprises a plurality of processor cores and a set of instructions executing on the plurality of processor cores to perform the exemplary methods discussed above. The processor cores or separate processor may be incorporated in or may communicate with any suitable electronic device, for example, on board processing arrangements within the device or processing arrangements external to the device, e.g., a mobile computing device, a smart phone, a computing tablet, a computing device, etc., that may be in communications with at least a portion of the device.

### EXAMPLES

### Example I

In Example I, the device and method of the present application may be used to detect and treat acne and/or pre-emergent acne. Redness of the skin or erythema of the skin caused by an infection or inflammation, such as acne or pre-emergent acne is correlated with an increase of redness from blood flow of the affect portion of skin. Therefore, the exemplary device of Example I is configured to detect an increase of biologic components for increased blood flow, such as, for example, an increased amount of oxyhemoglobin and deoxyhemoglobin at the affected portion of skin.

Light absorption differs across different wavelengths for different biologic components, such as, for example, brown eumelanin, black eumelanin, oxyhemoglobin, and deoxyhemoglobin. For example, both oxyhemoglobin and deoxyhemoglobin absorb less light within a red spectrum as compared to a green spectrum, and therefore, a red light and a green light can be used to differentially detect and/or measure levels of oxyhemoglobin and deoxyhemoglobin on the skin. In particular, a far-red light having a peak wavelength at or about 690 nm is absorbed significantly less by oxyhemoglobin and deoxyhemoglobin compared to a green light having a peak wavelength at or about 540 nm. Furthermore, oxyhemoglobin absorbs more red light than deoxyhemoglobin, but as the wavelength increases within an infrared spectrum, deoxyhemoglobin absorbs more light then oxyhemoglobin. Therefore, a combination of red and infra-red light may be used to detect oxyhemoglobin present in an area of skin and provide improved skin penetration to detect pre-emergent acne lesions. Brown eumelanin, black eumelanin and other skin colorings (e.g., skin bruising) can also be differentially detected and/or measured by red or far-red light compared to green light. A blue light can be further used to differentially detect and/or measure levels of oxyhemoglobin and deoxyhemoglobin on the skin with a green light. In particular, oxyhemoglobin and deoxyhemoglobin absorb blue light significantly more than green light, but brown eumelanin and black eumelanin absorbs more green light than blue light. Therefore, image data obtained using a blue light source, a green light source and/or a far-red light source in different combinations can be analyzed to identify redness of the skin or erythema of the skin and detect skin artifacts corresponding to acne or pre-emergent acne. Furthermore, different wavelengths of light may detect components from different depths of the skin, corresponding a level of skin penetration. Within the visible spectrum, as the wavelength increases the level of skin penetration generally increases. As the level of skin penetration continues to increase with wavelength until a peak level penetration is reached at about a wavelength of 1,100 nm providing about 3.5 mm of skin penetration. Therefore, near infra-red light allows for detection at a higher level of skin penetration (e.g., approximately twice) as compared to visible light. Although Example I describes detection of a acne and/or pre-emergent acne, it is contemplated that differences in light absorption across different wavelengths may be used to detect different components of the skin so as to identify other types of skin artifacts and/or diseases (e.g., skin cancer).

An exemplary device comprises a detector arrangement having three separate light sources: (1) a blue light source; (2) a green light source; and (3) a far-red light source. The blue light source has a peak wavelength at or about 450 nm. The green light source has a peak wavelength at or about 540 nm. The far-red light source has a peak wavelength at or about 690 nm.

In Example I, the detector arrangement of the exemplary device may be further configured to emit and detect polarized light from the skin to identify portions of skin having faint redness or erythema that is not otherwise visible to the human eye and provide early detection and treatment of acne. A tracer having a cyan color that absorbs far-red light is applied to the skin in proportional amounts to a benefit agent for treating acne in the manners discussed above in methods 200, 300. In particular, the tracer absorbs far-red light having a peak wavelength at or about 690 nm. Such an exemplary tracer absorbs light within the visible spectrum but is weakly visible to the human eye while imparting a slight green tint to modify appearance of redness or erythema of the skin caused by the acne.

### Example II

In Example II, another embodiment of the device and method of the present application for detecting and treating acne and/or pre-emergent acne is provided. An exemplary device of Example II comprises a detector arrangement having three separate light sources: (1) a green light source; (2) a far-red light source; and (3) an infra-red light source. The green light source has a peak wavelength at or about 540 nm. The far-red light source has a peak wavelength at or about 690 nm. The infra-red light source has a peak wavelength at or about 840 nm. As can be seen in FIG. 9, differential absorption of an infra-red light compared to a green light is even greater than differential absorption of a far-red light compared to a green light. Therefore, image data obtained using a green light source, a far-red light source and/or an infra-red light source in different combinations can be used, similar to Example I, to identify redness of the skin or erythema of the skin and detect skin artifacts corresponding to acne or pre-emergent acne. Averaged over this triplicate of light sources, skin has almost twice the penetration compared to Example I to better detect subcutaneous events like pre-emergent acne, with reduced interference from surface events.

A fluorescent tracer having an infra-red excitation wavelength is applied to the skin in proportional amounts to a benefit agent for treating acne in the manners discussed above in methods 200, 300. The tracer can be excited by an infra-red light, in particular, a light having a peak wavelength at or about 840 nm and can emit a light in the visible spectrum so that an amount of the tracer on the skin can be detected and measured by the detector arrangement.

### Example III

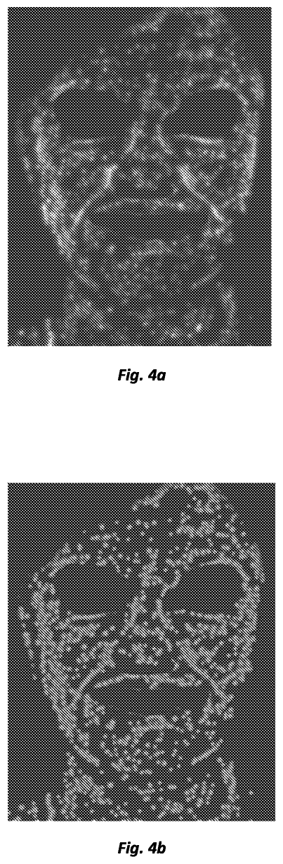

In Example III, the device and method of the present application may be used to detect and treat uneven skin tone. In this example, skin conditions that do not affect the melanin levels of the skin, such as infection, acne, wrinkles, etc. may be separately treated before utilizing the device and method of Example III to further improve skin tone. The exemplary device is configured to detect luminance nonuniformity and independently apply a first fixed dose of a skin brightening agent and a second fixed dose of a cosmetic composition when an artifact is detected, according to method 300. In particular, the device 100 utilizes illuminates an area of skin with a green light to detect luminance nonuniformity in the area of skin. It is believed that a green light is particularly useful for detecting luminance nonuniformity because green is a major contributor to the human eye's perception of uniformity. The skin brightening agent gradually alters the underlying skin tone by repeat usage over a period of time, while the cosmetic composition provides an immediate modification to the appearance of the skin. As the user moves the head portion of the exemplary device back and forth across the skin in multiple passes, the device applies the skin brightening agent until a predetermined threshold level is reached on a frexel of skin. The device applies the cosmetic composition based on the appearance of an artifact on the skin and is controlled independently from the skin brightening agent applied to the skin. Therefore, on frexels wherein the amount of skin brightening agent administered in a use session has reached the predetermined threshold level, the cosmetic may continue to be applied to the frexel until a desired appearance of the skin is reached. FIG. 4a shows a simulated image indicating location and amounts of a cosmetic composition applied to a face of a user over multiple passes across the face according to Example III. FIG. 4b shows a simulated image indicating location and amounts of a skin brightening agent applied to a face of a user over multiple passes across the face according to Example III.

### Example IV

In Example IV, the device and method of the present application may be used to detect and treat both acne and uneven skin tone. The exemplary device of Example IV is configured to apply two different compositions containing benefit agents: (1) a first composition comprising a benefit agent for treating acne; and (2) a second composition comprising a skin brightening agent. The first composition further comprising a fluorescent tracer that emits a green light, and the second composition further comprises a fluorescent tracer that emits a red light.

An exemplary device of Example IV comprises a detector arrangement having three separate light sources: (1) a blue light source; (2) a green light source; and (3) a far-red light source. The blue light source has a peak wavelength at or about 450 nm. The green light source has a peak wavelength at or about 540 nm. The far-red light source has a peak wavelength at or about 690 nm. The detector arrangement is configured to cycle through different combinations of the light sources and generate image data corresponding to images detected under these different combinations of light sources. Specifically, the detector arrangement cycles through two configurations of the light sources as follows: (1) the blue light and the green light in combination with the far-red light, and (2) the blue light without any other lights. The processing arrangement analyzes image data corresponding to those images obtained under the blue light to determine a first amount of the green fluorescent tracer and a second amount of the red fluorescent tracer detected from a frexel on the skin. The processing arrangement determines an amount of the first composition to apply based on a first magnitude of the portion of the artifact corresponding to redness of the skin or erythema of the skin and an amount of the green fluorescent tracer detected. The processing arrangement directs the applicator arrangement to apply the first composition until the amount of the green fluorescent tracer detected reaches a first predetermined threshold level. Similarly, the processing arrangement determines an amount of the second composition to apply based on a second magnitude of the portion of the artifact corresponding to a reduction in skin luminance and an amount of the red fluorescent tracer detected. The processing arrangement directs the applicator arrangement to apply the second composition until the amount of the red fluorescent tracer detected reaches a second predetermined threshold. The first predetermined threshold is determined independently from the second predetermined threshold level.

The invention described and claimed herein is not to be limited in scope by the specific embodiments herein disclosed since these embodiments are intended as illustrations of several aspects of this invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All publications cited herein are incorporated by reference in their entirety.

What is claimed is:

1. A handheld device for treating a skin condition, comprising:
an applicator arrangement applying a composition to skin, the composition comprising an active ingredient for treating the skin condition and a tracer;
a detector arrangement obtaining image data corresponding to an image of an area of skin; and
a processing arrangement analyzing the image data to determine whether an artifact corresponding to the skin condition is detected at a location within the imaged area of the skin and to determine an amount of the tracer detected from the location, the processing arrangement directing the applicator arrangement to apply the composition to the location when the artifact is detected and the amount of the tracer is less than a predetermined threshold level.

2. The device of claim 1, wherein the applicator arrangement is configured to apply a fixed dosage of the composition to the skin.

3. The device of claim 1, wherein the processor is configured to analyze the image data to determine an artifact magnitude of the artifact at the location, determine a dose of the composition to be applied to the location as a function of the artifact magnitude and the amount of the tracer, and direct the applicator arrangement to apply the dose of the composition to the location when the artifact is detected and the amount of the tracer is less than a predetermined threshold level.

4. The device of claim 1, wherein the predetermined threshold level of the tracer corresponds to a desired dosage threshold for the active ingredient.

5. The device of claim 1, wherein the detector arrangement comprises a first light source for delivering a far-red light or an infra-red light to the area of skin, and a sensor for detecting light from the area of skin.

6. The device of claim 5, wherein the far-red light has a peak wavelength of about 690 nm.

7. The device of claim 5, further comprising a second light source for delivering a green light, and a third light source for delivering an infra-red light.

8. The device of claim 5, wherein the second light source has a peak wavelength of about 540 nm, and the third light source has a peak wavelength of about 840 nm.

9. The device of claim 6, wherein the tracer absorbs light in a far-red spectrum and does not significantly absorb light in a visual red spectrum.

10. The device of claim 9, wherein the tracer is a cyan dye or a cyan pigment.

11. The device of claim 10, wherein the tracer is a non-sulfonated cyanine dye.

12. The device of claim 9, wherein the tracer absorbs light having a peak wavelength of about 690 nm and does not significantly absorb light having a peak wavelength of about 610 nm.

13. The device of claim 1, wherein the tracer is not visible to a human.

14. The device of claim 13, wherein the tracer is a fluorescent dye or a fluorescent pigment.

15. The device of claim 1, wherein the composition comprises at least one cosmetic ingredient for modifying an appearance of the skin.

16. The device of claim 15, wherein the composition comprises at least one of an opaque substance, a cosmetic pigment, and a cosmetic dye.

17. The device of claim 15, wherein the composition further comprises a reflectance modifying agent.

18. The device of claim 1, wherein the skin condition is selected from a group consisting of excessive melanin deposits, infection, inflammation, acne, wrinkles, and nonuniformities in skin color.

19. A method for treating a skin condition, comprising:
obtaining, by a detector arrangement, image data corresponding to an image of an area of the skin;
analyzing, by a processing arrangement, the image data to determine whether an artifact corresponding to the skin condition is detected at a location within the imaged area of the skin and to determine an amount of the tracer detected from the location; and
applying, by an applicator arrangement, a composition to the location when the artifact is detected from the location and the amount of the tracer is less than a predetermined threshold level, wherein the composition comprises an active ingredient for treating the skin condition and the tracer.

20. The method of claim 19, wherein the analyzing step comprises:
determining, by the processor, an artifact magnitude of the artifact at the location and the amount of the tracer detected from the location,
determining, by the processor, a dose of the composition to be applied to the location as a function of the artifact magnitude and the amount of the tracer detected, and
wherein the applicator arrangement applies the dose of the composition to the location when the artifact is detected and the amount of the tracer is less than a predetermined threshold level.
